(19) United States
(12) Patent Application Publication
(10) Pub. No.: US 2025/0082177 A1
(43) Pub. Date: Mar. 13, 2025

(54) DEGRADABLE VALVE FOR AN ENDOSCOPE

(71) Applicant: Boston Scientific Scimed, Inc., Maple Grove, MN (US)

(72) Inventors: Rosangel Aimee Ramos Espinoza, Allston, MA (US); Brian Luis, Boylston, MA (US); James J. Scutti, Norwell, MA (US)

(73) Assignee: Boston Scientific Scimed, Inc., Maple Grove, MN (US)

(21) Appl. No.: 18/826,946

(22) Filed: Sep. 6, 2024

Related U.S. Application Data
(60) Provisional application No. 63/581,041, filed on Sep. 7, 2023.

(51) Int. Cl. A61B 1/00; F16K 11/07; F16K 25/00

(52) U.S. Cl. CPC A61B 1/00068; F16K 11/07; F16K 25/005; F16K 2200/3051

(57) ABSTRACT

Devices, systems, and methods for a valve assembly for a medical device. The valve has a cap, a stem which moves within a valve body, and a spring member between the cap and the valve body to move the valve within the body. The valve stem and/or spring member are made of a degradable material, which may be a metal. The degradable material has a higher degradation rate than conventional metal valve components to reduce the environmental impact of disposing of the valve.

### CROSS-REFERENCE TO RELATED APPLICATIONS

This application claims the benefit of U.S. Provisional Patent Application Ser. No. 63/581,041 filed on Sep. 7, 2023, the disclosure of which is incorporated herein by reference.

### TECHNICAL FIELD

This disclosure relates generally to valve assemblies and methods, and particularly to supply valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

Some medical devices include components that are “single use,” intended to be discarded after a short window such as a single day or single procedure. These disposable components contribute significantly to the environmental costs associated with a procedure, as they are often categorized as biological waste. When a component is made of steel or another resilient material, the time and/or energy to break down the component can be significant. There is a need for medical device components that break down more easily to reduce the overall environmental burden associated with their disposal.

### SUMMARY

This disclosure provides design, material, manufacturing method, and use alternatives for medical devices and medical systems. In a first example, a valve assembly for a medical device can include a valve body having an air inlet passage, an air outlet passage, a water inlet passage, and a water outlet passage; a valve cap positioned above the valve body; a spring member positioned between the valve cap and the valve body such that, when the valve cap is pushed downward relative to the valve body, the spring member applies upward force against the valve cap; and a valve stem connected to the valve cap and configured to translate within the valve body between an upper position and a lower position, the valve stem comprising a side wall and a central lumen extending from an air inlet in the side wall of the valve stem to the air hole in the valve cap, the valve stem made entirely of a degradable metal.

Alternatively or additionally to any of the examples above, the degradable metal can comprise Mg. The degradable metal can be pure Mg or an alloy of Mg with a higher degradation rate than pure Mg.

Alternatively or additionally to any of the examples above, the degradable metal can comprise Zn. The degradable metal can be pure Zn or an alloy of Zn with a higher degradation rate than pure Zn.

Alternatively or additionally to any of the examples above, the degradable metal can comprise Fe. The degradable metal can be an alloy of Fe with a higher degradation rate than pure Fe.

Alternatively or additionally to any of the examples above, the spring member can be made entirely of a degradable metal.

In another example, a valve assembly for a medical device can include a valve body having an air inlet passage, an air outlet passage, a water inlet passage, and a water outlet passage; a valve cap positioned above the valve body; a spring member made entirely of a degradable material, the spring member positioned between the valve cap and the valve body such that, when the valve cap is pushed downward relative to the valve body, the spring member applies upward force against the valve cap; and a valve stem connected to the valve cap and configured to translate within the valve body between an upper position and a lower position, the valve stem comprising a side wall and a central lumen extending from an air inlet in the side wall of the valve stem to the air hole in the valve cap.

Alternatively or additionally to any of the examples above, the spring member can be a spring lever.

Alternatively or additionally to any of the examples above, the spring member can be a wavy washer stack.

Alternatively or additionally to any of the examples above, the spring member can be a disc washer stack.

Alternatively or additionally to any of the examples above, the degradable material can comprise at least one of Mg, Zn, and Fe.

Alternatively or additionally to any of the examples above, the degradable material can be pure Mg, an alloy of Mg with a higher degradation rate than pure Mg, pure Zn, an alloy of Zn with a higher degradation rate than pure Zn, or an alloy of Fe with a higher degradation rate than pure Fe.

Alternatively or additionally to any of the examples above, the valve stem can be made entirely of a degradable metal.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a perspective view of an illustrative supply valve;

FIG. 3A depicts a schematic cross-section view of an illustrative supply valve, with the valve in a first configuration;

FIG. 3B depicts a schematic cross-section view of an illustrative supply valve, with the valve in a second configuration;

FIG. 3C depicts a schematic cross-section view of an illustrative supply valve, with the valve in a third configuration;

FIG. 3D depicts a schematic cross-section view of an upper portion of an illustrative supply valve with a spring member;

FIG. 4A depicts a perspective view of an illustrative spring member;

FIG. 4B depicts a schematic cross-section view of an upper portion of illustrative supply valve with the spring member of FIG. 4A;

FIG. 5A depicts a perspective view of an illustrative spring member;

FIG. 5B depicts a schematic cross-section view of an upper portion of an illustrative supply valve with the spring member of FIG. 5A;

FIG. 6A depicts a perspective view of an illustrative spring member;

FIG. 6B depicts a schematic cross-section view of an upper portion of an illustrative supply valve with the spring member of FIG. 6A.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

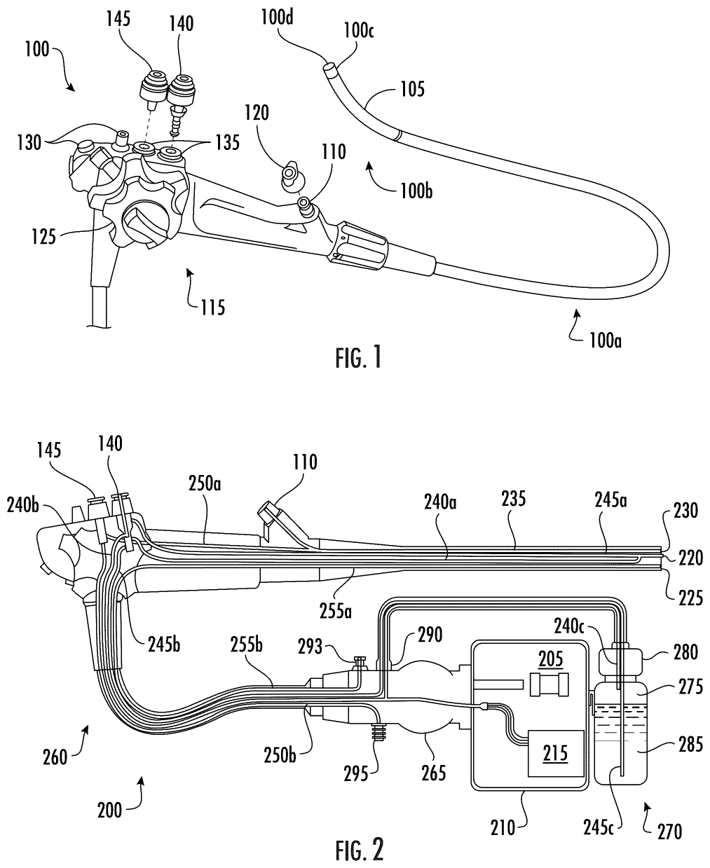

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

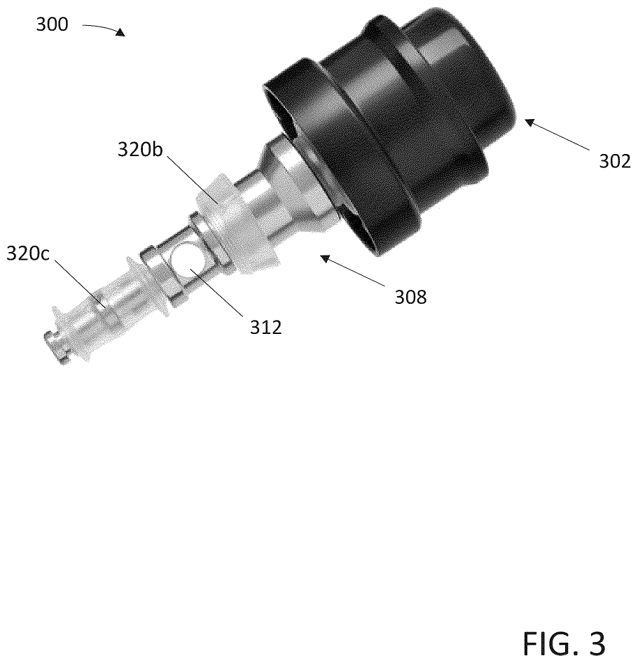

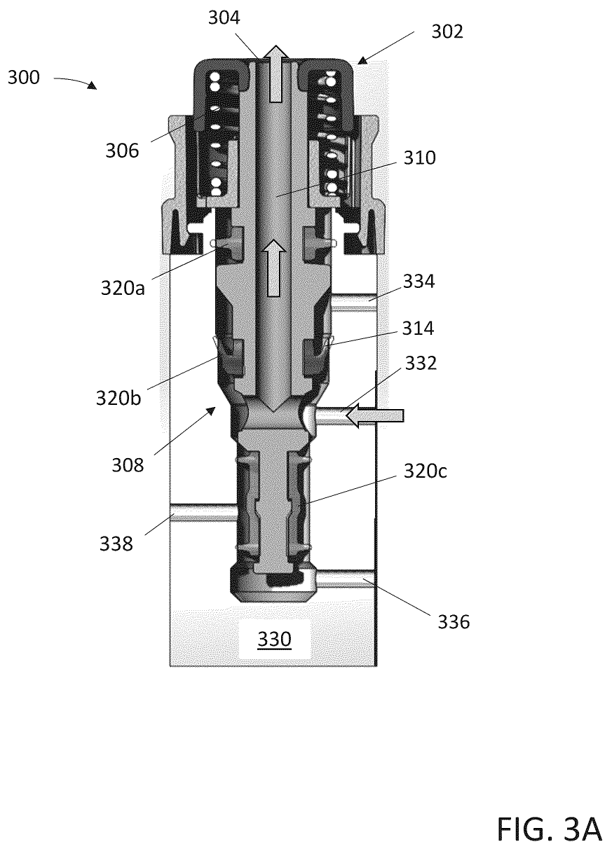

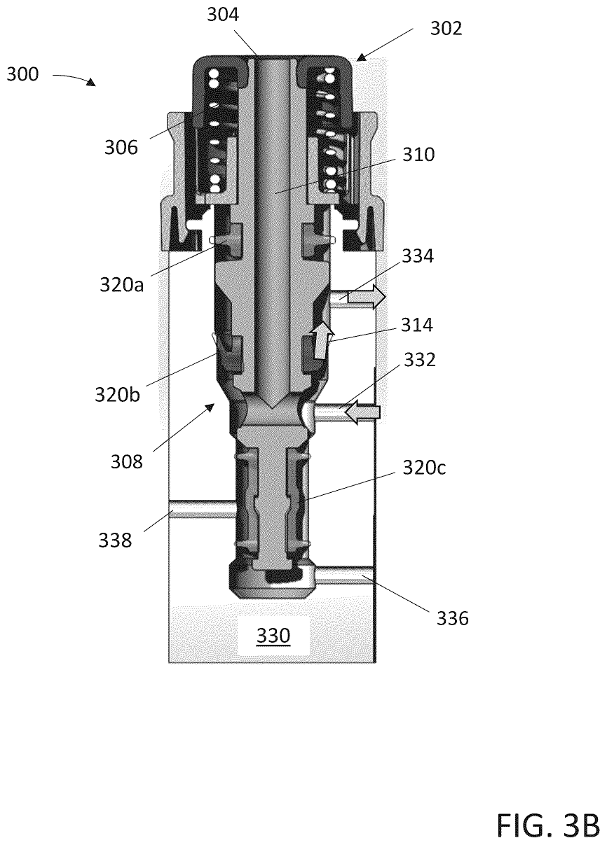

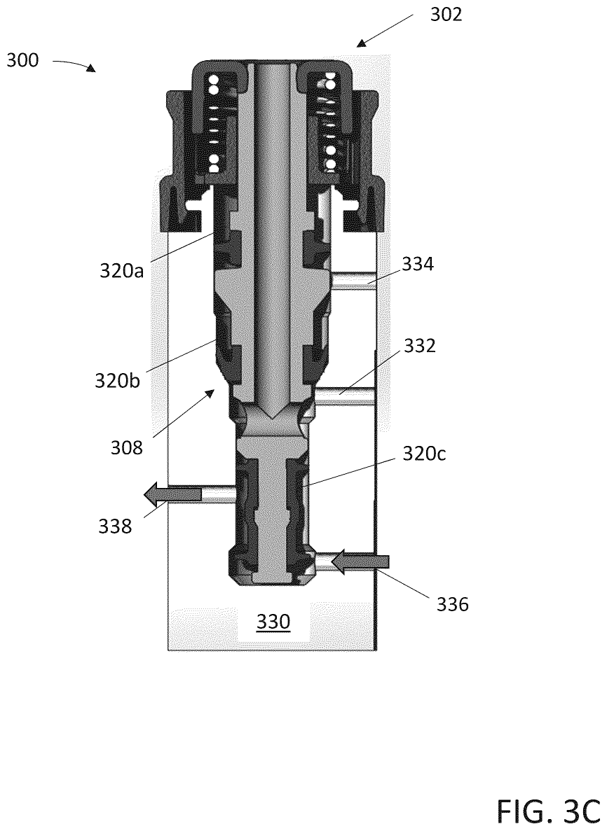

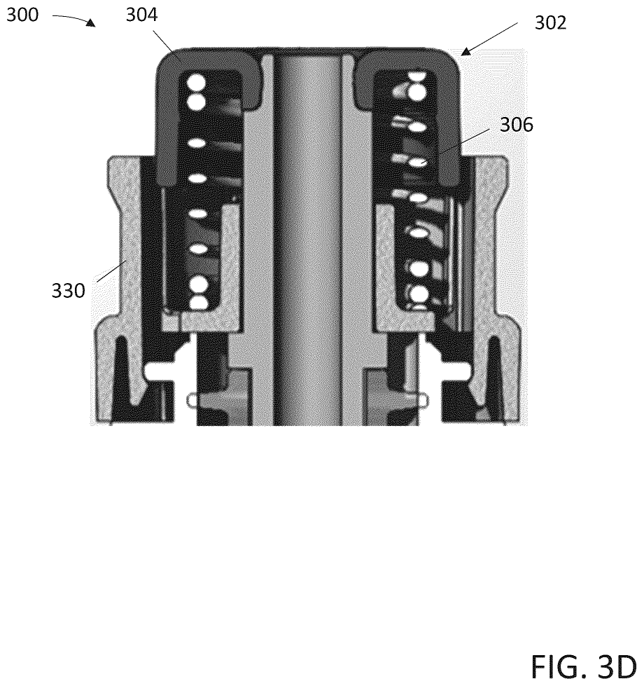

An example of a removable gas/water valve 300 is illustrated in FIG. 3 and in FIGS. 3A-3C. The valve cap 302 includes an air escape hole 304 and a spring member 306. The valve stem 308 includes a central lumen 310 connected to the air escape hole 304 and an air inlet 312.

The valve 300 is inserted into a body 330, such as one of the locations 135 described above and illustrated in FIGS. 1 and 2. The body 330 is sized and shaped to receive the stem 308 of the valve 300, as well as alternative valve designs (including each of those illustrated and described below). The valve body 330 includes an air inlet passage 332 communicating with a source of air as described above with respect to gas supply line 240a. An air outlet passage 334 similarly communicates with a gas feed line 240b.

FIG. 3A shows an open configuration for the valve when the air escape hole 304 is unblocked, air passing through the air inlet 312, up the central lumen 310, and out into the room. FIG. 3B shows a second configuration for the valve in which the air escape hole 304 is obstructed. In some implementations, the user may place a finger over the hole 304. A flap or other device may also be included for placement over the air escape hole 304 in other embodiments. When the air escape hole 304 is obstructed, air instead flows through a path defined by an exterior recess 314 in the valve stem 308 and the internal side wall of the body 330. Air passes through the air inlet passage 332, through the air outlet passage 334, and into the endoscope for use in insufflation as described.

Three seals 320a-c surround the valve stem 308 along its length, each including one or two wiper flanges configured to obstruct fluid flow when stationary without impeding vertical movement of the valve 300 within the body 330. An upper seal 320a is disposed below the valve cap 302 and above the exterior recess 314, obstructing flow in the valve well above the location of the air outlet passage 334. A middle seal 320b intersects the exterior recess 314 in the valve stem 308 but does not impede air flow when the valve 300 is in the upper position shown in FIGS. 3A and 3B. A lower seal 320c includes two wiper flanges.

The valve body 330 further includes a water inlet passage 336 connected to a water supply, and a water outlet passage 338 connected to a water feed line, at the lowest portion of the valve well. When the valve 300 is in the upper position as in FIGS. 3A and 3B, the lower of the two wiper flanges of the lower seal 320c sits above the water inlet passage 336, obstructing water from proceeding up the valve well or into the water outlet passage 338.

FIG. 3C shows a third configuration in which the valve 300 is positioned lower in the body 330. Downward force on the valve cap 302 causes and maintains this position; when the valve cap 302 is released, the spring member 306 returns the valve 300 to its previous position. In this configuration, the exterior recess 314 is no longer aligned with the air inlet 332 and air outlet 334 in the valve body 330. The middle seal 320b is seated along the inner wall of the body 330 to obstruct air flow above the air inlet 334. The two wiper flanges of the lower seal 320c are, in this configuration, positioned above the water outlet passage 338 and below the water inlet passage 336, creating an annular passage between the valve stem 308 and valve body 330 in which water can flow from the water inlet passage 336 to the water outlet passage 338. Upon release of the downward force and return of the valve cap 302 to its previous position, the placement of the lower seal 320c again prevents additional water from entering the feed through the annular passage. FIG. 3D shows the upper portion of the valve 300, in which the cap 302 interfaces with the valve body 330 by means of the spring member 306. The spring member 306 as shown is a coiled spring of steel wire that pushes against the valve body 330 to lift the valve 300 into the upper position.

The valve stem 308 may couple to the cap 302 in any suitable manner. In some cases, a portion (e.g., a proximal portion) of the valve stem 308 extending proximally of the air hole 304 may be coupled to the cap 302 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve stem 308 may have any suitable configuration configured to adjust positions within the valve well, adjust flow paths to the air and water supplies and feeds, and couple to the cap 302.

The valve 300 may be formed in any suitable manner. In some cases, though not required, the valve 300 may be formed using a molding process, an injection molding process, a casting process, a finishing process, sanding, and/or by or with one or more additional or alternative manufacturing techniques. In one illustrative example, the valve 300 may be formed using an injection molding process.

The valve stem 308 may be formed from a first material and the seals 320a-c may be formed from a second material, where the second material may be the same as or different than the first material. The valve stem 308 may be formed from a hard or rigid polymer and the seals 320a-c may be formed from a flexible polymer, but this is not required. The valve stem 308 would typically be formed from polymer, acrylonitrile butadiene styrene (ABS), or polycarbonate. Alternatively, the valve stem 308 could be formed of steel or aluminum. The seals 320a-c may be formed from one or more of a polymer, thermoplastic elastomers (TPE), thermoplastic polyurethane (TPU), liquid silicone rubber (LSR), and/or other suitable materials.

The material of the seals 320a-c may have any suitable durometer. In one example, the material of the seals 320a-c when formed on the valve stem 308 may have a durometer in a range of about 20-80 shore A, about 30-60 shore A, and/or other suitable values within one or more other suitable ranges of durometer, but could be softer or firmer depending on the geometry used for the seals and the amount of interference desired with the inner wall of the valve body 330. In one example, the seals 320a-c may be formed from silicone with a durometer in a range of 40-50 shore A, but this is not required.

In this specification, we define “degradation” as physical and/or chemical changes to a component that occur over time due to the component's exposure to an environment. Weathering, corrosion, and decomposition are three common examples of degradation.

Steel and aluminum are often chosen as materials for mechanical devices because of the metals' longevity and resistance to degradation. Moreover, particular alloys of steel and/or aluminum are often favored over other alloys due to superior resistance to degradation.

As an alternative, however, metals can be chosen to specifically have a higher degradation rate—that is, the metals will degrade in the same environment in a shorter period of time. Magnesium, zinc, and iron alloys are known to break down more quickly and have therefore been used as biodegradable materials for temporary insertion into human bodies. Their higher degradation rates also make alloys of these metals good candidates for reducing the environmental impact of disposable components. Both metal and non-metal materials that have a higher degradation rate are “degradable” materials.

The degradation properties of Mg, Zn, and Fe, as well as alloys of each of these, have been examined. A discussion of many of these materials can be found in Li et al, “Progress of biodegradable metals,” Progress in Natural Science: Materials International 24 (2014) 414-422, which is herein incorporated by reference in its entirety. Other alloys are known to those skilled in the art.

With respect to magnesium, the degradation rates of pure magnesium and alloys such as Mg—Zn—Mn, Mg—Ca, Mg—Sr, Mg—Si, Mg—Zr, AZ91D, AZ31, LAE442, and WE43 may make some or all of them appropriate substitutes for slower-degrading materials, along with other alloys currently known in the art, or later-discovered alloys having similar properties.

With respect to iron, the degradation rates of alloys such as Fe-3C, Fe-3S, Fe-3W, Fe-10Mn, Fe-10Mn-1Pd, Fe-30Mn (forged), Fe30Mn (cast), Fe-30Mn-1C, and Fe-30Mn-6Si may make some or all of them appropriate substitutes for slower-degrading materials, along with other alloys currently known in the art, or later-discovered alloys having similar properties.

With respect to zinc, the degradation rates of pure zinc and alloys such as Zn—Mg, Zn—Mg—Ca, Zn—Mg—Sr, Zn—Al, Zn—Mn, Zn—Ca, Zn—Sr, and Zn—Ag may make some or all of them appropriate substitutes for slower-degrading materials, along with other alloys currently known in the art, or later-discovered alloys having similar properties.

Returning to the valve 300 described and shown above, in some implementations, the valve stem 308 may be made of a degradable metal. The difference in the hardness, stiffness, and the resilience in degradable metal alloys from that of steel or aluminum will not meaningfully impede the functioning of the stem 308 as described above. For a chosen alloy, if the degradation rate upon exposure to water is faster than acceptable for a particular procedure or duration of use, the surface of the valve stem 308 may be coated with another substance, such as a degradable polymer. In some implementations, PHA or a similar biobased hydrocarbon polymer may be used.

In some implementations, the spring member 306 may be made of a degradable material, which may be a degradable metal as described above or may be a degradable polymer. In some implementations, the degradable spring member may have a significantly lower strength and/or modulus than steel wire; therefore, a different geometry for the member may be used.

Degradable spring levers, wave washer stacks, and disc washer stacks described. These spring members may be used with a degradable material that is less ductile than steel and more difficult to manufacture as a coil spring, or where a coil spring or similar structure made of the degradable material would not have a sufficient spring constant to allow for easy operation of the valve. For example, a coil spring made of degradable thermoplastic may not have a spring constant sufficient to consistently move the valve as required, while a degradable thermoplastic spring lever provides sufficient force. As another example, a degradable iron alloy might not be sufficiently ductile for standard techniques to form it into a coil spring, but the degradable iron alloy may be efficiently stapled into a disc washer. Each spring member may be molded, stamped, tooled, drawn, rolled, cut and/or any manufacturing technique or combination techniques appropriate for the material and structure.

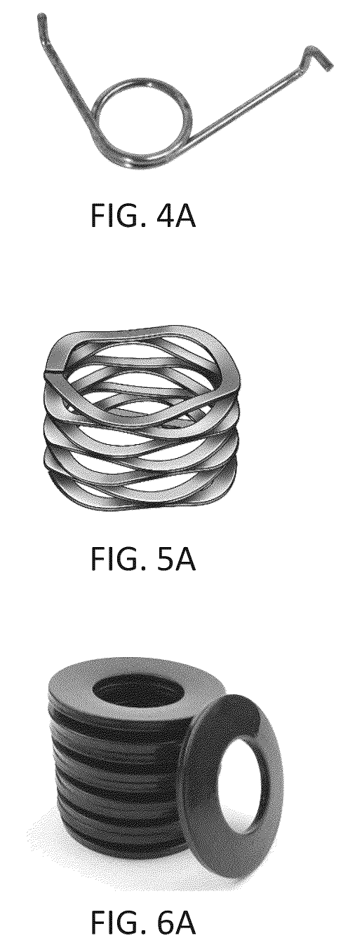

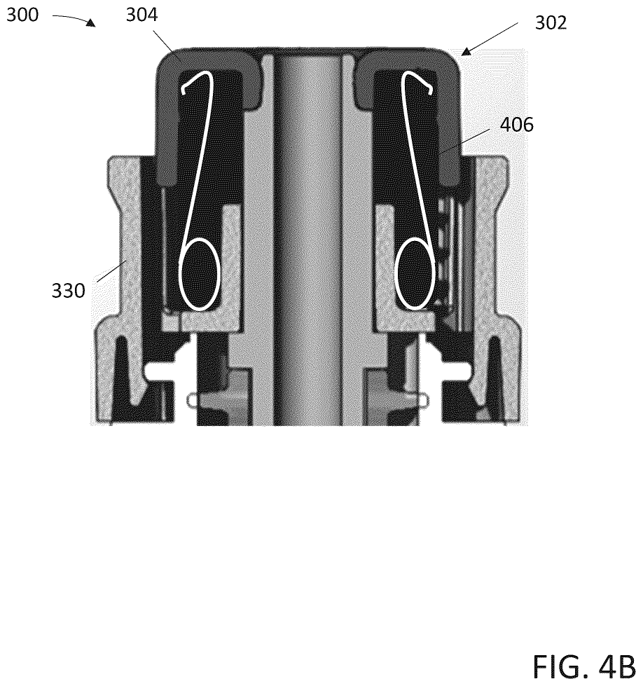

FIGS. 4A and 4B show a degradable spring member in the form of a spring lever 406. A plurality of spring levers 406 may be included around the circumference of the cap 302. As with other versions of the spring member, the spring levers 406 may be attached or affixed to the cap 302 in various manners known in the art. The spring levers 406 may flex when the valve 300 is moved downward against the valve body 330, pushing upward to restore the valve 300 to the upper position. The spring levers 406 may be made of a degradable polymer such as thermoplastic with a high degradation rate. The spring levers 406 may also be made of a magnesium, zinc, or iron alloy, or any other degradable metal.

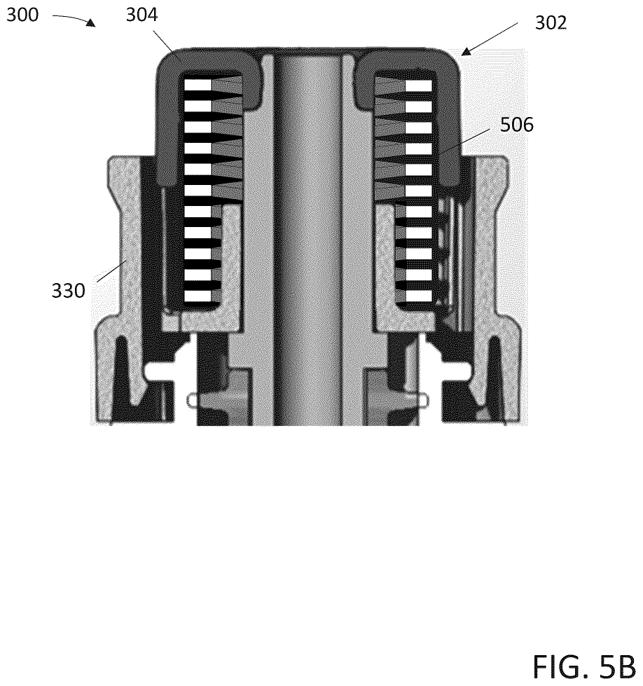

FIGS. 5A and 5B show a degradable spring member in the form of a wave washer stack 506. The wave washer stack 506 may be made of a plurality of wave washers of similar shape, but with the upper and lower portions of the waves offset to contact and provide the basis for restorative force when the stack is compressed. Pressing downward on the valve cap 302 compresses the wave washer stack 506, which exerts upward force on the cap 302 when released to move the valve 300 back into the upper position. The wave washer stack 506 may be made of a degradable polymer such as thermoplastic with a higher degradation rate. The stack 506 may also be made of a magnesium, zinc, or iron alloy, or any other degradable metal.

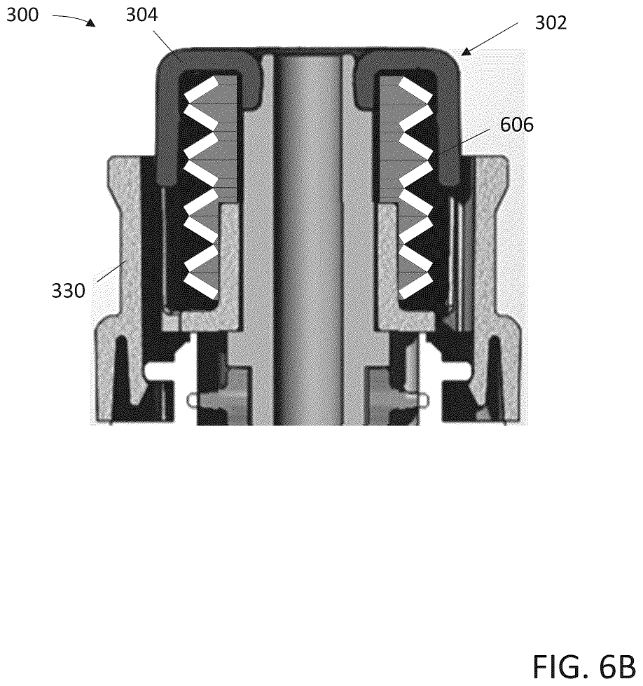

FIGS. 6A and 6B show a degradable spring member in the form of a disc washer stack 606. The disc washer stack 606 may be made of a plurality of disc washers, each of which is similarly shaped as a truncated cone or a truncated sphere (sometimes referred to as “Belleville” washers). The stacked disc washers provide a combined spring force that is approximately linear, a sum of the individual spring forces of the washers. Pressing downward on the valve cap 302 compresses the disc washer stack 606, which exerts upward force on the cap 302 when released to move the valve 300 back into the upper position. The disc washer stack 606 may be made of a degradable polymer such as thermoplastic with a higher degradation rate. The stack 606 may also be made of a magnesium, zinc, or iron alloy, or any other degradable metal. The overall diameter of the washers, as well as the distance that the inner diameter of ach the washer is raised to form the truncated conical shape, and the corresponding angle above the horizontal of each washer, can be chosen according to the properties of the degradable material and the requirements of the resulting valve.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve assembly for a medical device, comprising:
a valve body having an air inlet passage, an air outlet passage, a water inlet passage, and a water outlet passage;
a valve cap positioned above the valve body;
a spring member positioned between the valve cap and the valve body such that, when the valve cap is pushed downward relative to the valve body, the spring member applies upward force against the valve cap; and
a valve stem connected to the valve cap and configured to translate within the valve body between an upper position and a lower position, the valve stem comprising a side wall and a central lumen extending from an air inlet in the side wall of the valve stem to the air hole in the valve cap, the valve stem made entirely of a degradable metal.

2. The valve assembly of claim 1, wherein the degradable metal comprises Mg.

3. The valve assembly of claim 2, wherein the degradable metal is pure Mg or an alloy of Mg with a higher degradation rate than pure Mg.

4. The valve assembly of claim 1, wherein the degradable metal comprises Zn.

5. The valve assembly of claim 2, wherein the degradable metal is pure Zn or an alloy of Zn with a higher degradation rate than pure Zn.

6. The valve assembly of claim 1, wherein the degradable metal comprises Fe.

7. The valve assembly of claim 2, wherein the degradable metal is an alloy of Fe with a higher degradation rate than pure Fe.

8. The valve assembly of claim 1, wherein the spring member is made entirely of a degradable metal.

9. A valve assembly for a medical device, comprising:
a valve body having an air inlet passage, an air outlet passage, a water inlet passage, and a water outlet passage;
a valve cap positioned above the valve body;
a spring member made entirely of a degradable material, the spring member positioned between the valve cap and the valve body such that, when the valve cap is pushed downward relative to the valve body, the spring member applies upward force against the valve cap; and
a valve stem connected to the valve cap and configured to translate within the valve body between an upper position and a lower position, the valve stem comprising a side wall and a central lumen extending from an air inlet in the side wall of the valve stem to the air hole in the valve cap.

10. The valve assembly of claim 9, wherein the spring member is a spring lever.

11. The valve assembly of claim 9, wherein the spring member is a wavy washer stack.

12. The valve assembly of claim 9, wherein the spring member is a disc washer stack.

13. The valve assembly of claim 9, wherein the degradable material comprises Mg.

14. The valve assembly of claim 13, wherein the degradable material is pure Mg or an alloy of Mg with a higher degradation rate than pure Mg.

15. The valve assembly of claim 9 wherein the degradable material comprises Zn.

16. The valve assembly of claim 15, wherein the degradable material is pure Zn or an alloy of Zn with a higher degradation rate than pure Zn.

17. The valve assembly of claim 9, wherein the degradable material comprises Fe.

18. The valve assembly of claim 17, wherein the degradable material is an alloy of Fe with a higher degradation rate than pure Fe.

19. The valve assembly of claim 9, wherein the degradable material is a polymer with a high degradation rate.

20. The valve assembly of claim 9, wherein the valve stem is made entirely of a degradable metal.
